(19) United States
(12) Patent Application Publication
(10) Pub. No.: US 2006/0235729 A1
(43) Pub. Date: Oct. 19, 2006

(54) Application-specific biometric templates

(76) Inventors: Michael Braithwaite, Langhorne, PA (US); Ulf Cahn von Seelen, Radnor, PA (US); James L. Cambier, Medford, NJ (US); John G. Daugman, Huntington (GB); Randal Glass, Cherry Hill, NJ (US); Russell Moore, Sewell, NJ (US); Ian Scott, Haddonfield, NJ (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/453,959

(22) Filed: Jun. 14, 2006

Related U.S. Application Data
(63) Continuation of application No. 09/860,991, filed on May 18, 2001, now abandoned.

(51) Int. Cl. G06F 19/00; A61B 5/00

(52) U.S. Cl. 705/3

(57) ABSTRACT

Disclosed are techniques for transforming a biometric template so that each application uses a unique template format. One transformed template cannot be successfully matched to a second template extracted from the same biologic entity unless the second template is transformed so that its format is identical to that of the first template. Thus a template generated in a format corresponding to application A could not be used to authenticate a user for application B because the enrollment database for application B would have a different format than the enrollment database for application A.

### FIELD OF THE INVENTION

The present invention relates generally to systems and methods for using biometric data to authenticate identity. More particularly, the invention relates to protecting access to personal biometric information through the use of transformation functions so that each application has a unique biometric template format.

### BACKGROUND

In biometric authentication, a human or animal biological entity (e.g. finger, hand, eye, voice, etc.) is measured. Information unique to that individual is extracted and encoded in a standard data format called a biometric template. The initial extraction of biometric information and storage of that information in a database is called “enrollment”. To establish or verify identity, biometric information is extracted anew and a “recognition” template is generated and compared to one or more enrollment templates in the enrollment database.

Biometric data may be supplemented with secondary identification information such as name, address or identification number. The database is indexed by the secondary information, so that the user's enrollment template can be easily retrieved from a database. The recognition and enrollment templates are compared and, if a match is found, the user's identity is confirmed. Matching a recognition template to a single enrollment template that is retrieved from a database indexed by a secondary identifier is called “verification”.

In “identification” systems, secondary identifying information is not required to retrieve a specific enrollment template from a database. The recognition template is compared against all templates in an enrollment database. An index or identification number may be stored with each enrollment template, however, to link that template to individual identification or privilege information contained in a separate database. When an identification attempt is successful, the index or identification number of the matching enrollment template is typically returned or reported so it can be used in granting privileges. Identification is practical only if the biometric technology employed is extremely accurate and specific, so that false matches rarely occur.

A verification or identification system containing a large database of enrollment templates enables the establishment of a centralized authentication server, for use by a number of applications. Applications include maintaining physical security, information security, financial transactions, testing services, voter registration, immigration, entitlements, and so on.

Access to biometric databases by multiple applications raises data privacy concerns because biometric templates can be considered to be personal information that can be used for unauthorized purposes such as fraud. For example, stolen enrollment templates could be used to misrepresent personal identity. Furthermore, once a biometric template is compromised, it cannot be re-issued like a password can. Hence the theft of conventional biometric data is irreversible.

The iris recognition technology described in U.S. Pat. No. 4,641,349 (Flom et al.), U.S. Pat. No. 5,291,560, (Daugman), and U.S. Pat. Nos. 5,572,596 and 5,751,836 (Wildes et at.), provides a powerful recognition capability, using a standard biometric template format. Cryptographic techniques can be used to protect biometric data that is stored in various types of digital media. Techniques to protect integrity and privacy of digital data, including biometric data, are known to those skilled in the art. A specific technique is described in co-pending application Ser. No. 09/232,538 entitled “Method and Apparatus for Securely Transmitting and Authenticating Biometric Data Over a Network,” which is hereby incorporated by reference. One approach is to encrypt templates, but because the algorithms used to match templates, and thereby authenticate individual identity, cannot typically operate on encrypted templates, the templates must be decrypted prior to matching, exposing the decrypted template to attacks during the matching process. Furthermore, cryptographic algorithms can be computationally expensive and can have resulting deleterious effects on system performance.

Thus, techniques for protecting access to personal biometric information that overcomes the drawbacks of the prior art is needed.

### SUMMARY OF THE INVENTION

The present invention discloses systems and methods for transforming a biometric template so that each application has a unique format. One transformed template cannot be successfully matched to a second template extracted from the same biologic entity unless the second template is transformed so that its format is identical to that of the first template. Thus a template generated in a format corresponding to application A could not be used to authenticate a user for application B because the enrollment database for application B would have a different format than the enrollment database for application A. The ability to create changeable, unique formats for biometric templates allows users to replace or re-issue biometric data that has been compromised.

### BRIEF DESCRIPTION OF THE DRAWINGS

The foregoing summary, as well as the following detailed description of preferred embodiments, is better understood when read in conjunction with the appended drawings. For the purpose of illustrating the invention, there is shown in the drawings exemplary constructions of the invention; however, the invention is not limited to the specific methods and instrumentalities disclosed. In the drawings:

FIG. 1a is a flow diagram of an enrollment portion of a biometric authentication method as is well-known in the art;

FIG. 1b is a flow diagram of a recognition portion of a biometric authentication method as is well-known in the art;

FIG. 2a is a flow diagram of an exemplary enrollment portion of an exemplary biometric authentication method in accordance with one aspect of the invention;

FIG. 2b is a flow diagram of an exemplary recognition portion of an exemplary biometric authentication method in accordance with one aspect of the invention;

FIG. 3 is a flow diagram of an exemplary biometric authentication method in accordance with an aspect of the invention, wherein a template is transferred to another database;

FIG. 4 is a flow diagram of an exemplary biometric authentication method in accordance with an aspect of the invention, wherein an authorization template authenticates a transfer of a template to another database;

FIG. 5 is a flow diagram of an exemplary biometric authentication method in accordance with an aspect of the invention, wherein a unique key is used to authenticate a transfer of a template to another database;

FIG. 6 is a flow diagram of an exemplary biometric authentication method in accordance with an aspect of the invention, wherein a user template is generated using a second transformation function; and

FIG. 7 is a block diagram of an exemplary computing environment in which aspects of the invention may be implemented.

### DETAILED DESCRIPTION OF THE INVENTION

Overview

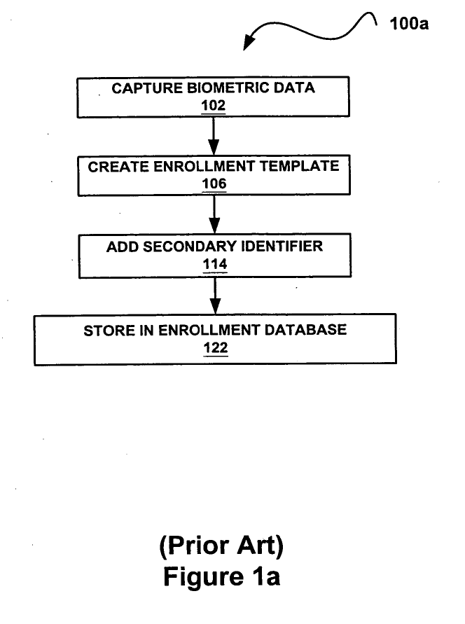

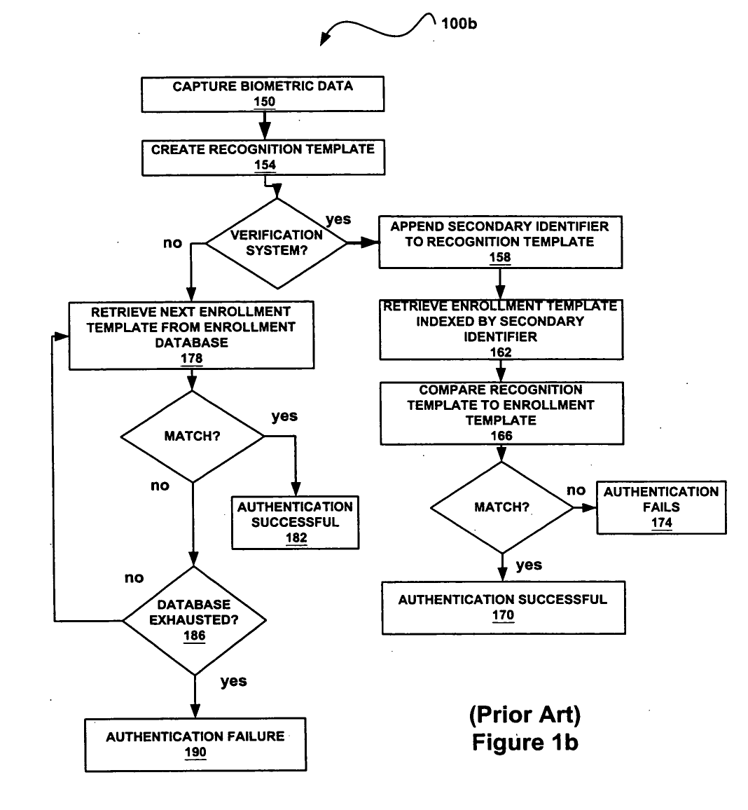

FIG. 1a represents a portion of a typical biometric authentication technique 100a as is well-known in the art, in which enrollment data is captured and stored in a database. Referring now to FIG. 1a, at step 102 biometric data is captured, using methods that are well-known to those of skill in the art. At step 106, the biometric data is encoded into a biometric template, using methods well-known to those skilled in the art. Processing proceeds to step 114, where secondary identification information such as name, address, or identification is stored. In verification systems, this information is concatenated to the biometric template and both are stored in a biometric database. In identification systems, the secondary information is typically stored in a separate secondary information database. An appropriate database key value, such as an index number or identification number, is concatenated to the biometric template and is stored in a separate template database. A separate template database for identification is used to permit optimized, high-speed searches of the database as part of the identification matching process. When a matching template is found its concatenated identification number or database key is then used to retrieve the corresponding information from the secondary information database. At step 122 the biometric data and secondary information is stored in an enrollment database. The database may be indexed by the secondary identification information.

FIG. 1b represents a recognition portion of a typical biometric authentication technique 100b as is well-known in the art. At step 150, biometric data is captured. At step 154, a recognition template is created using methods well-known to those skilled in the art. At step 158, if the system is a verification system, secondary information is appended to the template. At step 162 the enrollment template for the user, as identified by the secondary identifier, is retrieved from the database of enrollment templates. At step 166, the enrollment template and the recognition template are compared. At step 170 if the recognition template matches the enrollment template, authentication is successful. At step 174, if the recognition template does not match the enrollment template, authentication fails.

If the system is an identification system the recognition template is compared with a template in the enrollment (template) database. At step 182, if the enrollment template and the recognition template match, authentication is successful. If the templates do not match, at step 186, the system checks to see if there are more templates in the database. If there are more templates in the database, processing returns to step 178 and the next template in the database is retrieved, and the process is repeated. If all the templates have been compared to the recognition template and no match has been found, authentication fails (step 190).

Application-Specific Biometric Templates

The present invention discloses systems and methods for transforming a biometric template so that each application that uses a biometric template to control access to the application, is associated with a unique template format. One transformed template cannot be successfully matched to a second template extracted from the same biologic entity unless the second template is transformed so that its format is substantially identical to that of the first template. Thus a template generated in a format corresponding to application A could not be used to authenticate a user for application B because the enrollment database for application B would have a different format than the enrollment database for application A.

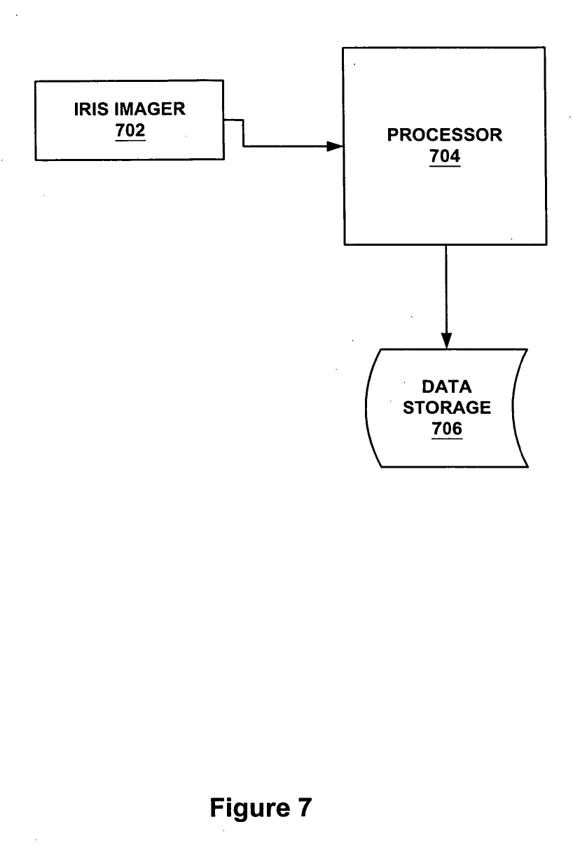

FIG. 7 depicts an exemplary computer environment in which aspects of the present invention may be implemented. An iris imager 702 is coupled to a processor 704 to which is coupled storage 706. An image of a user's iris is captured by iris imager, 702. Iris imager transmits the iris image to a processor 704. Processor 704 processes the iris image and compares the resultant template to a database of stored templates. Examples of well known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, wireless devices, distributed computing environments that include any of the above systems or devices, and the like.

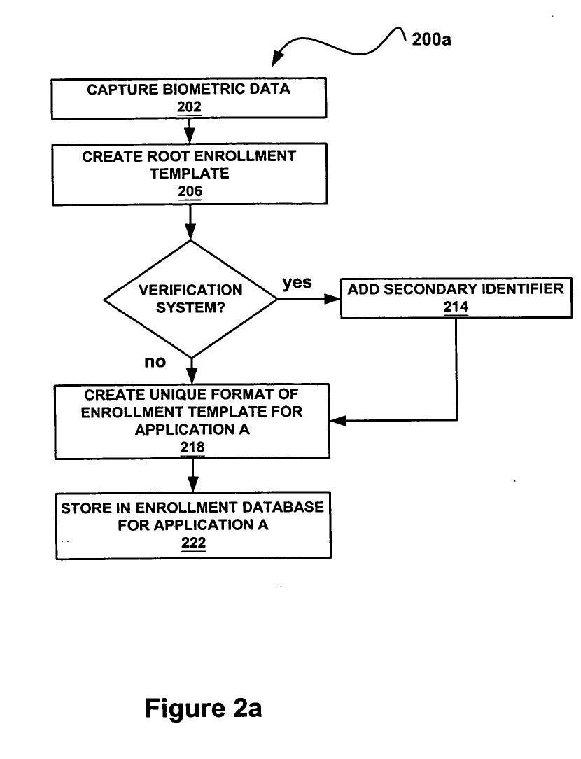

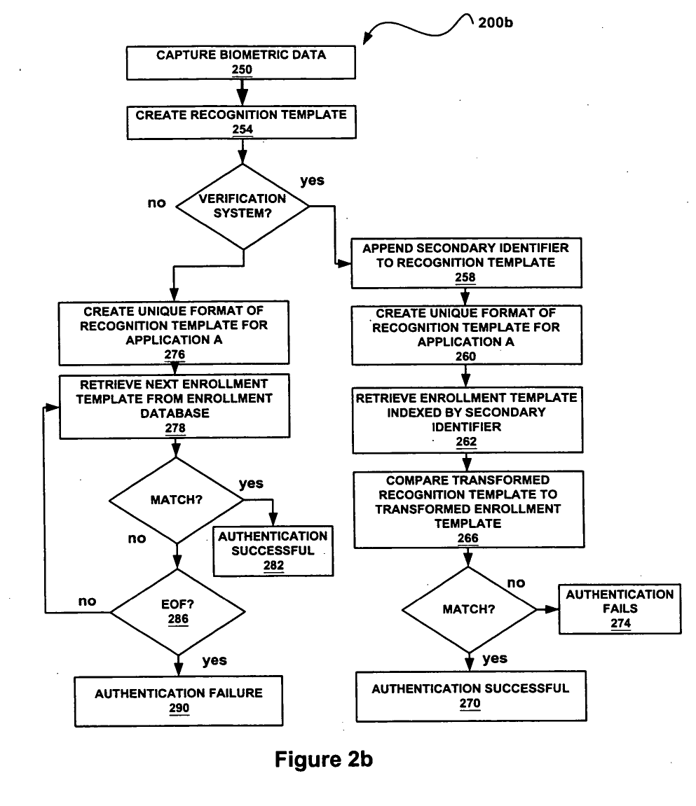

FIG. 2a represents a flow diagram of an exemplary enrollment portion of a biometric authentication method 200a in accordance with one aspect of the present invention. The enrollment process 200a creates a database for an application, where the database contains enrollment templates having a format unique to the application. In method 200a biometric data from the user is processed to create a root enrollment template having a standard format. The root template is then transformed using a transformation function so that the format of the transformed template is specific to a particular application. An enrollment database of transformed templates for a particular application is generated as transformed templates are added to the database.

For example, and referring now to FIG. 2a, at step 202, biometric data is captured, using processes that are well-known to those skilled in the art. At step 206, a root enrollment template T₁ for user 1 is created. If the system is a verification system, as described above, processing proceeds to step 214. At step 214, secondary identification information such as name, address or identification is associated with the biometric template such as by concatenation. At step 218, a transformation function F_{A} for an application A is applied to the root enrollment template, T₁ with the resultant transformed template being represented by

F_{A} (T₁). At step 222, the resultant transformed template F_{A} (T₁) is then stored in a database DB_{A} where DB_{A} is the database of transformed enrollment templates for application A. The database DB_{A} may be indexed by secondary identification information in a verification system.

The transformed template F_{A} (T₁) is unique for application A so that F_{A}(T₁) preferably will not successfully match with any other application, (such as for example, application B), even if root enrollment template T₁ or is the root template for both applications. Likewise F_{B} (T₁) preferably will not successfully match with application A.

FIG. 2b represents a flow diagram of an exemplary recognition portion of a biometric authentication method 200b in accordance with one aspect of the present invention, in which a root recognition template is created and compared to a database of transformed enrollment templates for a particular application. The root recognition template is captured using methods well-known to those skilled in the art and transformed using a unique transformation function for the application. A matching function (described below) compares the transformed recognition template with one or more transformed templates from the enrollment database for the application. If a match is found, the authentication process is successful. If no match is found, the authentication process fails. The matching function compares the transformed recognition template with one (if the system is a verification system or more (if the system is an identification system) transformed enrollment templates from the application database.

For example, and referring now to FIG. 2b, at step 250, biometric data of a user 1 desiring access to application A is captured, using methods that are well-known to those skilled in the art. At step 254, a recognition template T₁ is created using methods well-known to those skilled in the art. At step 258, if the system is a verification system, secondary information is appended to the template. At step 260 the transformation function F_{A} for application A is applied to the root recognition template. At step 262 the transformed enrollment template for the user, as identified by the secondary identifier, is retrieved from the database of enrollment templates for the application. At step 266, the enrollment template and the recognition template are compared using a matching algorithm such as one described below. At step 270 if the recognition template matches the enrollment template, authentication is successful. At step 274, if the recognition template does not match the enrollment template, authentication fails.

If the system is an identification system, a database key value, index, or identification number is appended to the biometric template. At step 276, the transformation function F_{A} for application A is applied to the root recognition template, T₁, with the resultant transformed template being represented by F_{A} (T₁). At step 278, the recognition template is compared with each template in the enrollment database until a match is found. At step 282, if a match is found, authentication is successful and an index, database key, or identification number is returned for use in retrieving corresponding secondary identification information from the secondary identification database. In an identification system such an index or database key is required unless all individuals in the enrollment database have identical privileges. Such a system is described in co-pending application entitled “Anonymous Biometric Authentication”, U.S. application Ser. No. 09/781,733. If no match is found for the recognition template, at step 286, the system determines if there are more templates in the database. If there are more templates in the database, the next template is retrieved at step 278 and the process is repeated. If all the templates in the database have been compared to the recognition temple and no match has been found, authentication fails (step 290).

It should be understood that although the example illustrates the generation of a single enrollment template, a plurality of templates may be generated, representing a plurality of samples of the same biometric entity, thus accounting for variation in the template generation process which may otherwise result in false rejections of the recognition template.

According to another aspect of the invention, the transformed enrollment and recognition template could be created directly, without ever generating the root template, by incorporating the transformation process into the template generation process, thus avoiding possible exposure of the root template to piracy.

A. The Matching Algorithm

A matching algorithm preferably compares at least two transformed templates. A determination is made as to whether the templates being compared came from the same biological entity. As stated above, the transformed template F_{A} (T₁) is unique for application A so that F_{A} (T₁) will not successfully match with templates from any other application, such as for example, application B, even if root enrollment template T₁ is the root template used for both applications. Likewise F_{B} (T₁) will not successfully match with transformed templates for application A.

For example, consider biometric templates T₁, and T₂ derived from the same biologic entity (e.g. hand, finger, eye, etc.) so that an appropriate matching function M(T₁, T₂) has a value:
M(T₁,T₂)=1
if the templates match (i.e. they came from the same biologic entity) and
M(T₁,T₂)=0
if the templates do not match. If templates T₁ and T₂ are generated in the same way with the same format and come from the same biologic entity, preferably M(T₁, T₂) will have a value of 1, meaning that a match has been found.

According to one aspect of the invention, a transformation function F_{A} applied to the root templates T₁ and T₂ creates transformed templates F_{A}(T₁) and F_{A}(T₂), having a unique format specific to application A. It is preferable that the transformation F_{A} have the property that the matching process is invariant under the transformation, that is:
M(F_{A}(T₁),F_{A}(T₂))=M(T₁,T₂)
This invariance is desirable because it means that matching can be performed on the transformed templates, making it unnecessary to reverse the transformation, thereby recreating and exposing the root templates T₁ and T₂ prior to or during the matching process.
B. Properties of Transformation Functions

A template generated in a format corresponding to application A cannot be used to authenticate a user for application B because the enrollment database for application B has a different format than the enrollment database for application A. For example, if the transforming function for application A is F_{A} and the transforming function for application B is F_{B}, then as stated previously, comparison of the transformed template for application A with the transformed template for application B for the same biometric sample, will not be successfully authenticated. In mathematical terms:
M(F_{A}(T₁),F_{B}(T₂))=0
where T₁ and T₂ are root biometric samples from the same biological entity. This property assures that a template generated for one application A cannot be used for another application B.

However, in contrast, if the transformation function for application A is applied to both root biometric samples from the same biological entity, it is preferable that authentication is successful, or in mathematical terms:
M(F_{A}(T₁),F_{A}(T₂))=1 and
M(F_{B}(T₁),F_{B}(T₂))=1

If a template from Application A were used to attempt to authenticate to a database created for Application B, authentication fails. The user template is created with the format of Application A, while all the enrollment templates have the format of Application B. Preferably, the match function, when comparing templates with different formats, will nearly always return a zero, indicating no match. The probability of such a match returning a value of one will be no greater than the likelihood of two randomly selected templates matching, which is to say the likelihood will be no greater than the single-match false-accept probability of the biometric technology. In the case of exceptionally strong biometric technologies like iris recognition, this probability is extremely small. This is true even if the two templates T₁ and T₂ are from the same biologic entity and even if T₁ and T₂ are identical. Preferably, a template with format corresponding to F_{A} will in general not match any template in the enrollment database of application B even if that database contains an enrolled template from the same biologic entity. Hence templates enrolled for application A, preferably, cannot be sold, stolen, licensed, or in other ways misappropriated to authenticate to Application B, or to create or expand an enrollment database for Application B because their format will be incompatible.

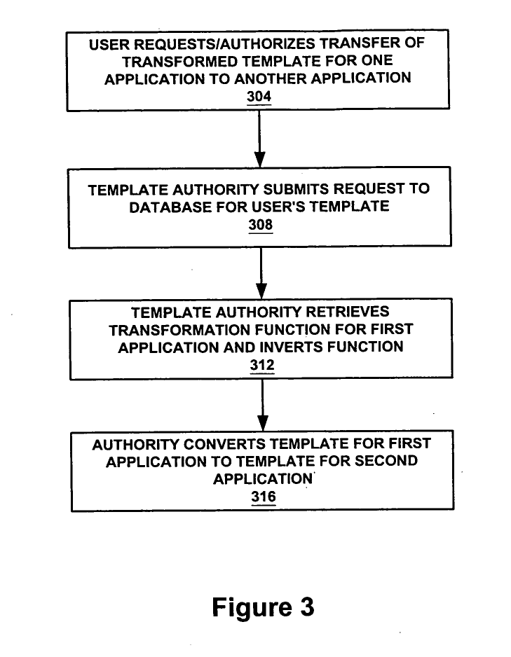

According to another aspect of the invention, as shown in FIG. 3, existing format transformations can be processed to create new templates. For example, if template F_{A}(T₁) exists, transformation F_{A,B} can be created, such that applying the transformation function F_{A,B} for application B onto a transformed template for application A will result in a transformed template for application B, or in other words:
F_{B}(T₁)=F_{A,B}(F_{A}(T₁))
or
F_{A,B}=F_{B}F_{A}⁻¹
where F_{B} is the format created for application B and F_{A}⁻¹ is the inverse of transformation A, having the property that:
F_{A}(F_{A}⁻¹(T))=T.
If user 1 has created an enrolled template for application A, user 1 can authorize the custodian of database DB_{A} to make the user 1's enrolled template F_{A}(T₁) available to the application B database, DB_{B} after application of transformation F_{A,B} to F_{A}(T₁) to change the format of the application A-transformed template.

In this case, preferably, responsibility for definition and application of transformation F_{A,B} can rest in a trusted format authority that maintains a registry of formats and defines and applies the transformations desired to convert templates from one format to another.

As shown in FIG. 3, at step 304 user 1 requests and authorizes the transfer of user 1's existing enrollment template, created for application A, to the enrollment database for application B. At step 408 a Template Authority submits a (preferably) authenticated request to application A database, DB_{A} for user 1's enrolled template, that exists in the database DB_{A} in a format consistent with application A. Upon receiving user 1's template, at step 312 the Template Authority retrieves application A's transformation function F_{A} (e.g. from archival storage), inverts it, and then converts the result at step 316 to Application B's format by applying the Application B format F_{B}. According to this aspect of the invention, an application transformation is not exposed to another application, and yet users may be able to use their existing enrollments for new applications without incurring the cost and inconvenience of re-enrolling their biometric for each new application.

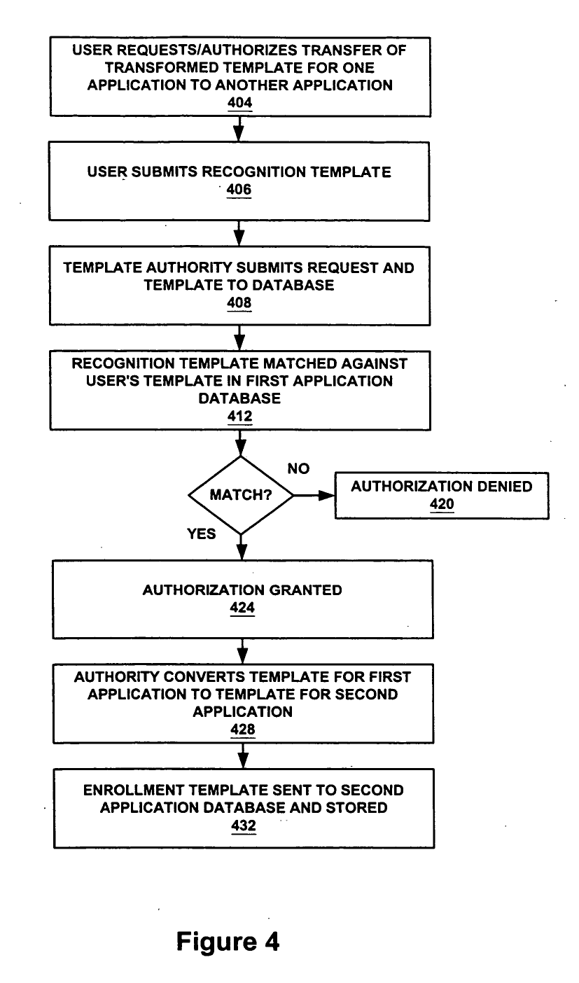

Preferably, such transformations would be performed only if specifically requested and authorized by the user who produced the original template. According to one aspect of the invention the biometric itself is used to authorize the transfer of the enrollment template as shown in FIG. 4.

At step 404 user 1 submits a request for transfer of user 1's enrollment template for application A (F_{A}(T₁)) from application A to application B. User 1 also submits a recognition template (F_{A}(T₂)) as evidence of authorization to the Template Authority at step 406. At step 408, the Template Authority submits the data request, along with user 1's recognition template, (F_{A}(T₂)) to the application A database DB_{A}. At step 412, the recognition template (F_{A}(T₂)) is matched against the template (verification system) or templates (identification system) of the application A database DB_{A}. If the Matching function is unsuccessful, the transfer is denied at step 420. If authorized, at step 424, user 1's enrollment template (F_{A}(T₁)) from the database for application A, DB_{A} is returned to the Template Authority. At step 428, the template authority creates and applies the appropriate transformation F_{B}F_{A}⁻¹ to convert user 1's enrollment template (F_{A}(T₁)) to the application B format. At step 432, the enrollment template F_{A,B}(F_{A}(T₁)) is transmitted to the application B database, DB_{B} and stored in database DB_{B}.

Preferably, the database owner of application A database, DB_{A} has no knowledge of the format of application B database DB_{B} and vice versa. Preferably, both the transforms and their inverses are secret. Preferably, the format authority can control the transfer of templates from one database to another, avoiding the inconvenience and substantial cost of constant re-enrollments as biometric applications proliferate, yet protecting the privacy of individual users by protecting the templates and transformations.

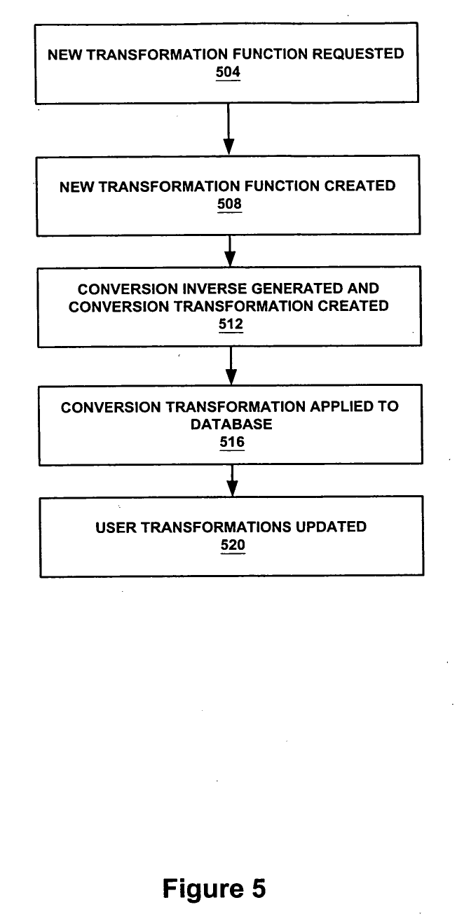

In accordance with another aspect of the invention, and as illustrated in FIG. 5, if the custodian of a database suspects or determines that biometric data in the database has been compromised, or the format of the data has been discovered, the Template Authority is requested to define a new transformation function for the database. Preferably, by changing the format of the templates in the compromised database, the stolen templates are rendered invalid.

Referring now to FIG. 5, at step 504 a request is sent from application A for a new format. At step 508, the Template Authority creates a transformation function F_{C} that will be the new transformation function for Application A. At step 512, using the (preferably archived) transformation function for Application A, F_{A}, the Authority generates the inverse of F_{A} and processes F_{A} with F_{C} to form F_{C}F_{A}⁻¹ called the conversion transformation. At step 56 the conversion transformation F_{C}F_{A}⁻¹ is applied to the application A database, DB_{A}, to convert application A's enrollment templates to the new format, generated by function F_{C}. At step 520 all of user transformations are updated to reflect the change in format from that produced by F_{A} to that produced by F_{C}.

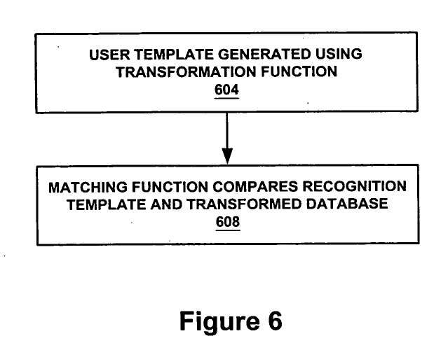

FIG. 6 illustrates an exemplary authentication process using the new transformed database DB_{C} for Application A. At step 604, a user template is generated using the transformation function F_{C}. At step 608, matching, as discussed above, is performed against the application A database, now containing enrollment templates having the “C” format.

Preferably, such a capability provides a powerful defense against loss or theft of biometric templates, either through observation of the transmission of templates across a network, or by penetration of an enrollment database. Optionally, periodic database transformation may be applied to existing databases so that if data is stolen, the stolen template will remain valid only until the next transformation is applied.

Authentication may be required in a client-server environment in which the user, running a client application, wishes to request a service (such as an electronic transaction) from a server application running on a different computer. The client and server computers may be interconnected through a local or wide area network. It is well known that replay attacks can be used in such a system, in which authentication data transmitted over a network is observed and recorded by an attacker and then replayed later in an attempt to gain access to the legitimate user's privileges. A defense against such attacks is the application of a “single use” transformation, that is only valid for a single transaction between the server and any client. In accordance with another aspect of the invention, a user whose converted template F_{A}(T₁) has been stored in Application A database DB_{A}, initiates such a transaction by requesting an authentication server for a unique, single-use transformation number or transformation key. The authentication server may generate a random or otherwise unique number or key X. The server may transmit the unique number or key X to the client and approximately simultaneously applies a transformation function where the unique key X is part of the transformation function. In other words:
F_{X,A}=F_{A} Fₓ⁻¹
The transformed template F_{X,A} (T₁) is saved, preferably in temporary storage. The unique key, the transformation function using the unique key X, F_{X}, and the inverse of F_{X}, F_{X}⁻¹ are deleted. The client upon receiving X, generates the function F_{X}. A root biometric template T₁ is then captured. The root biometric template T₁ is transformed using transformation function F_{X}, creating F_{X}(T₁). The transformed template F_{X}(T₁) is digitally signed using digital signature generating procedures that are well-known to those who are skilled in the art. The transformed template F_{X}(T₁) may optionally be encrypted or signed and encrypted. The signed and/or encrypted template is transmitted to the server. The server decrypts the template, if the template was encrypted, and verifies the integrity of the template using standard digital signature techniques. The server uses the preferably temporarily-stored transformation function F_{X,A} to convert the user's template to a format compatible with application A database, DB_{A}. In other words:

$$\begin{matrix}{F}_{A} ( {T}_{1} ) = {F}_{X , A} ( {F}_{X} ( {T}_{1} ) ) \\ = {F}_{A} {F}_{X}^{- 1} ( {F}_{X} ( {T}_{1} ) )\end{matrix}$$

Thus, the client's template has been generated and transmitted to the server in a unique format valid for only a single transaction. Only the server has the information needed to render F_{X}(T₁) compatible with the enrollment database, DB_{A}.

In accordance with another aspect of the invention, before the enrollment process is performed, the client application generates a unique transformation function F_{A}. The client then creates a unique A transformation function F_{A}. Transformation function F_{A} is applied to the root enrollment template before the template is sent to the server. The transformation function F_{A}, or information required to generate it may also be stored on a smart card or other form of portable media that the user may keep in his possession. This aspect of the invention enables the user to perform enrollments for a number of applications, each time saving the appropriate transformation in portable storage. Each template in the enrolled database will have its own unique format, known only to the user, thus enabling the user to have complete control over the use of the user's biometric data. The unique format of the biometric template is defined by the transformation stored on the portable media.

When authentication for application A is required, the user may capture an image with the appropriate biometric device and generate a root template. The user may then insert the portable media for the A application into an appropriate reader. Such devices are well-known in the art. The client application may read in the transformation function, and apply the transformation funciton to the root template. The transformed template may be sent to the server. It should be noted that, as previously discussed, the transformed template may be encrypted and digitally signed prior to sending to the server.

C. Data Structure for Biometric Templates

In one embodiment of the invention, a biometric template may include an array [t₁ t₂ t₃ . . . tₙ] of independent data entities tᵢ, where tᵢ may be isolated binary bits or groups of bits. In one embodiment of the invention, the matching function is one that judges the similarity between two templates by examining corresponding independent data entities. An exemplary matching function is the function known as the Hamming Distance function, HD(T₁, T₂). The Hamming Distance function examines every pair of corresponding bits in templates T₁ and T₂ and counts the proportion of bits that differ between the two templates. The HD concept can be generalized to larger data entities, counting the number of corresponding entities that are not identical. For example, bits might be examined in groups of 2 bits, in which one bit represents a data value and the second bit a control bit indicating the validity of the data bit. In this case, the two data bits are compared and used in the HD calculation only if both control bits have a value confirming the validity of the data bits.

A preferred transformation function for an application A, F_{A} used for transforming biometric templates in accordance with the present invention preferably does not alter the length of the template, change the value of the control bits or alter the number of matching (or mismatching) data bit pairs. A preferred transformation is permutation, that alters the position of some or all data bits. For a template including n independent entities, there are n! possible transformations. For example, if the data entities are 8-bit bytes, and there are 256 data bytes in each template, the number of possible permutations is 256!=8.6×10⁵⁰⁶. If the data entities are single bits, the number of permutations is 2048! that is approximately 10⁵⁸⁹⁴. In one embodiment of the invention only transformations that alter the position of every data entity, are used, preventing the possibility of false matches. Such permutations are termed “derangements”. The number of possible derangements of 256 data elements, for example, is 6.2×10⁵⁰⁶. All such permutations possess readily-computed inverses.

Another form of transformation is based on the logical exclusive-or (XOR) function. In this transformation single bit values are XORed with a predefined mask function. If Tᵢ is the ith data bit of template T and Mᵢ is the ith mask bit then the ith transformed template bit is:
Fᵢ(T)=Tᵢ XOR Mᵢ
The XOR function changes the value of any bit for which the corresponding mask bit is a 1. If the template has 2048 data bits, for example, the number of possible masks is 2²⁰⁴⁸=3.2×10⁶¹⁶. Preferably, the mask contains 1's in at least half its positions to avoid ineffective transformations that do not significantly affect the template. The number of such transformations is 1.6×10⁶¹⁶. The XOR function serves as its own inverse.

It is also possible to combine transformations of different types. Thus a permutation could be followed by a logical XOR transformation, further enhancing the security of the templates and increasing the number of possible forms of transformation. The extremely high number of possible, unique transformations of the biometric template makes the scheme highly effective against brute force attacks.

It is noted that the foregoing examples have been provided merely for the purpose of explanation and are in no way to be construed as limiting of the present invention. While the invention has been described with reference to various embodiments, it is understood that the words which have been used herein are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

1. A method for securing access to protected applications, the method comprising:
receiving a predetermined biometric template;
transforming said predetermined biometric template to generate a transformed biometric template having a unique format corresponding to a particular biometric application, the transforming comprising at least one of altering the position of every bit of data of the biometric template using derangement permutations and exclusive-oring the data of the biometric template with a mask function;
storing said transformed biometric template in a storage;
receiving a first biometric template;
transforming said first biometric template using a first transforming function to create a first transformed biometric template, the first transformed biometric template having the unique format corresponding to the particular biometric application; and
comparing said first transformed biometric template with said stored transformed biometric template.

2. The method of claim 1, wherein said transforming said first biometric template to create a first transformed biometric template further comprises applying a first transforming function to said first biometric template to create said first transformed biometric template.

3. The method of claim 1, wherein said comparing further comprises determining that said first transformed biometric template is approximately equal to said transformed biometric template.

4. The method of claim 3 further comprising enabling access to a protected application responsive to said comparing.

5. The method of claim 1, further comprising indexing said transformed template by a key.

6. The method of claim 1, further comprising creating a second transformed biometric template by applying a second transforming function to said first biometric template.

7. The method of claim 6, wherein said second transformed biometric template is not approximately equal to said first transformed biometric template created by applying said first transforming function to said first biometric template.

8. The method of claim 1, further comprising receiving a second transformed biometric template created from a second transforming function, wherein said second transformed biometric template is not approximately equal to said first transformed biometric template.

9. The method of claim 1, further comprising generating a plurality of biometric templates corresponding to one entity.

10. The method of claim 1, wherein said transforming function comprises:
receiving data unique to a user; and
transforming said data into a transformed biometric format by applying an encoding and transforming function to said data.

11. The method of claim 6, wherein said second transforming function further comprises:
receiving said first transformed biometric template in a first format;
transforming said first transformed biometric template in said first format into said second transformed biometric template in a second format by applying said second transforming function.

12. The method of claim 11, wherein said second format is not approximately equal to said first format.

13. The method of claim 1, wherein at least one of a plurality of transforming functions is maintained by a template authority.

14. The method of claim 13, wherein said at least one of a plurality of transforming functions is maintained in secret.

15. The method of claim 11, further comprising receiving a request for transformation of a transformed template from said first format to said second format.

16. The method of claim 15, further comprising receiving the transformed template as authorization to convert said first transformed template into said second transformed template.

17. The method of claim 11, wherein a plurality of said first transformed templates in said first format are transformed periodically into a plurality of said second transformed templates of said second format.

18. The method of claim 6, wherein said second transforming function incorporates a secret transformation key.

19. A system for securing access to protected applications comprising:
a receiver for receiving a biometric template;
computer-executable instructions for:
receiving a predetermined biometric template;
transforming said predetermined biometric template to generate a transformed biometric template having a unique format corresponding to a particular biometric application, the transforming comprising at least one of altering the position of every bit of data of the biometric template using derangement permutations and exclusive-oring the data of the biometric template with a mask function;
storing said transformed biometric template in a storage;
receiving a first biometric template;
transforming said first biometric template using a first transforming function to create a first transformed biometric template, the first transformed biometric template having the unique format corresponding to the particular biometric application; and
comparing said first transformed biometric template with said stored transformed biometric template; and
a database for storing said transformed biometric template templates.

20. The system of claim 19, wherein said computer-executable instructions further comprise instructions for determining that said first transformed biometric template is approximately equal to said transformed biometric template.

21. The system of claim 20, wherein said computer-executable instructions further comprise instructions for enabling access to a protected application responsive to the comparing.

22. The system of claim 19, wherein said computer-executable instructions further comprise creating a second transformed biometric template by applying a second transforming function to said first biometric template.

23. The system of claim 22, wherein said second transformed biometric template is not approximately equal to said first transformed biometric template created by applying a first transforming function to said first biometric template.

24. The system of claim 19, further comprising a receiver for receiving a second transformed biometric template created from a second transforming function wherein said second transformed biometric template is not approximately equal to said first transformed biometric template.

25. The system of claim 19, wherein a plurality of biometric templates are generated corresponding to one entity.

26. The system of claim 19, further comprising a receiver for receiving data unique to a user.

27. The system of claim 26, further comprising computer-executable instructions for transforming said data into a transformed biometric format by applying an encoding and transforming function to said data.

28. The system of claim 24, further comprising:
a receiver for receiving said first transformed biometric template in a first format; and
computer-executable instructions for transforming said first transformed biometric template in said first format into said second transformed biometric template in a second format by applying said second transforming function.

29. The system of claim 28, wherein said second format is not approximately equal to said first format.

30. The system of claim 28, further comprising a template authority.

31. The system of claim 30, wherein said template authority maintains said first and second transforming functions in secret.

32. The system of claim 28, wherein the receiver is adapted to receive a request for transformation of a transformed template from said first format to said second format.

33. The system of claim 19, wherein the receiver is adapted to receive the transformed template as authorization to convert said first transformed template into said second transformed template.

34. The system of claim 28, further comprising computer-executable instructions for periodically transforming a plurality of transformed templates in said first format into a plurality of second transformed templates in said second format.

35. The method of claim 34, wherein said second transforming function incorporates a secret key.

36. A computer-readable medium comprising computer-executable instructions for performing the method of claim 1.
